(19) United States
(12) Patent Application Publication
(10) Pub. No.: US 2022/0241499 A1
(43) Pub. Date: Aug. 4, 2022

(54) PERISTALTIC INFUSION PUMP TUBE SEGMENT AND INFUSION PUMP DEVICE WITH SUCH A TUBE SEGMENT

(71) Applicant: Micrel Medica Devices S.A., KOROPI (GR)

(72) Inventor: Achilleas Tsoukalis, Papagou (GR)

(73) Assignee: Micrel Medica Devices S.A., KOROPI (GR)

(21) Appl. No.: 17/665,152

(22) Filed: Feb. 4, 2022

(30) Foreign Application Priority Data
Feb. 4, 2021 (EP) 21020053.1

(51) Int. Cl. A61M 5/168; A61M 5/142; A61M 5/00; A61M 5/165; F04B 43/08

(52) U.S. Cl. CPC A61M 5/16854; A61M 5/14228; A61M 5/002; A61M 2205/3375; F04B 43/08; A61M 2005/16872; A61M 2005/16868; A61M 5/165

(57) ABSTRACT

A peristaltic infusion pump tube segment comprises a first tube portion having a surrounding wall adapted for an upstream occlusion detection, a second tube portion downstream of said first tube portion and adapted to be temporally engaged by an engagement structure, wherein said second tube portion comprises a surrounding wall being at least partly thicker than the wall of said first tube portion and made of an elastic material which allows said second tube portion to be essentially reformed at those portions which are currently not subject to the engagement by the engagement structure, a third tube portion downstream of said second tube portion and having a surrounding wall adapted for an air in line detection, and a fourth tube portion adapted for a downstream pressure detection. The peristaltic infusion pump tube segment is provided in an infusion pump device which comprises a pump mechanism configured as a peristaltic mechanism.

### RELATED APPLICATIONS

This application claims priority to EP Patent Application No. 21020053.1, filed on Feb. 4, 2021, which is incorporated herein by reference in its entirety.

### BACKGROUND OF THE INVENTION

Ambulatory infusion pumps are used outside and inside the hospital, for smaller volume drug delivery like 100 ml morphine to palliative care patients, or higher volume drug delivery like in case of parenteral nutrition where large volumetric pumps (LVP) are too big to carry. A big use of ambulatory infusion pumps is with chronic patients at home or with therapies that last long with the patient at home.

False alarms of infusion pumps are very problematic but frequent. It is the aim of the present invention to provide means to reduce false alarms.

Accuracy of infusion pumps is far lower than required, since mostly PVC tubes are used which show the required accuracy for 30 minutes after start of infusion but deteriorate largely after several hours or days of infusion. It is the aim of the present invention to increase and keep the required accuracy of the pump very long after start of infusion.

### BRIEF DESCRIPTION OF THE INVENTION

In order to achieve the aforementioned and further objects, according to a first aspect of the present invention, there is provided a peristaltic infusion pump tube segment having an upstream inlet and a downstream outlet and adapted to be temporally squeezed at at least one squeezed point to be generated by an engagement structure and to be moved towards said outlet for passing a medical fluid from said inlet to said outlet, characterized by a first tube portion next to said inlet and having a surrounding wall which is adapted for an upstream occlusion detection, a second tube portion downstream of said first tube portion and adapted to be temporally engaged by the engagement structure, wherein said second tube portion comprises a surrounding wall being at least partly thicker than the wall of said first tube portion, a third tube portion downstream of said second tube portion and having a surrounding wall which is adapted for an air in line detection, and a fourth tube portion next to said outlet and adapted for a downstream pressure detection.

Preferred embodiments and modifications of the first aspect of the present invention are defined in the dependent claims 2 to 11.

Preferably, the peristaltic infusion pump tube segment is, at least partly, made of silicone, in particular by a silicone molding process, preferably by a silicone injection molding process.

According to a further preferred embodiment of the first aspect, the wall of said first tube portion has a thickness which is dimensioned so as to allow a detection unit to detect even the smallest possible upstream occlusion.

According to a further preferred embodiment of the first aspect, the wall of said second tube portion is made of an elastic material which allows said second tube portion to be essentially reformed at those portions which are currently not subject to the engagement by the engagement structure.

According to a further preferred embodiment of the first aspect, the wall of said second tube portion is configured to allow a viscous fluid to be passed through by suction effect.

According to a further preferred embodiment of the first aspect, the wall of said second tube portion is essentially unpolished.

According to a further preferred embodiment of the first aspect, the wall of said third tube portion comprises at least one flat wall portion which is configured for the air in line detection in particular by means of at least one ultrasound sensor.

According to a further preferred embodiment of the first aspect, said third tube portion has a rectangular or square section at least at its external side and comprises flat wall portions configured for the air in line detection in particular by means of at least one ultrasound sensor.

According to a modification of both the aforementioned preferred embodiments, the outer surface of the at least one flat wall portion is at least partly polished.

According to a further preferred embodiment of the first aspect, said fourth tube portion comprises an essentially flat compartment configured for the downstream pressure detection.

According to a preferred modification of the above embodiment, said flat compartment comprises a long side extending in the direction of the peristaltic infusion pump tube segment.

According to a further preferred modification of the above embodiment, said flat compartment comprises a disc-like element having essentially an at least part-circle or part-elliptical shape with a diameter or width being larger than the diameter or width of said first tube portion and/or said second tube portion and/or said third tube portion.

In order to achieve the aforementioned and further objects, according to a second aspect of the present invention, there is provided an infusion pump device comprising a pump mechanism module configured as a peristaltic mechanism and including the peristaltic infusion pump tube segment according to the first aspect, wherein the upstream inlet of said peristaltic infusion pump tube segment is adapted to be fluidly connected to an outlet of a medication reservoir.

Preferred embodiments and modifications of the second aspect are defined in the dependent claims 13 to 23.

According to a preferred embodiment of the second aspect, said pump mechanism module is configured as a linear peristaltic mechanism with said peristaltic infusion pump tube segment having an essentially elongated shape.

According to a further preferred embodiment of the second aspect, said peristaltic infusion pump tube segment is replaceable and/or disposable.

According to a further preferred embodiment of the second aspect, the infusion pump device comprises an upstream pressure sensor provided at said first tube portion.

According to a further preferred embodiment of the second aspect, said pump mechanism module comprises an engagement structure adapted to generate at least one squeezed point in said second tube portion and to move it in the direction towards said outlet of said peristaltic infusion pump tube segment.

According to a further preferred embodiment of the second aspect, said engagement structure comprises a plurality of engagement units arranged side by side along the length of said second tube portion of said peristaltic infusion pump tube segment, wherein each engagement unit comprises a follower head which is movable at an angle, in particular a right angle, relative to said second tube portion and adapted to be brought into engagement with said second tube portion in order to squeeze it, wherein said engagement structure is further configured so that the follower heads are temporally brought into engagement with said second tube portion one after another.

According to a modification of the above embodiment, at least one engagement unit comprises a support, a follower body moveably mounted at said support and provided with the follower head, a ball bearing rotatably mounted at said follower body, a cam rotatably mounted at said support and in sliding engagement with said ball bearing, and a spring biasing said moveable follower body with said follower head away from said second tube portion of said peristaltic infusion pump tube segment.

According to a further modification of the above embodiment, said engagement structure comprises a drive unit, wherein the cam of each equipment unit is fixedly mounted to a common rotary shaft which is rotated by said drive unit, and wherein the rotational angle offset of the cam relative to said rotary shaft increases from engagement unit to engagement unit so that the follower heads are temporally brought into engagement with said second tube portion of said peristaltic infusion pump tube segment from engagement unit to engagement unit.

According to a still further modification of the above embodiment, the follower head is mounted upon a stem fixed to the follower body and the engagement unit further comprises a sealing membrane with an opening through which said stem extends in an at least essentially sealing manner.

According to a further preferred embodiment of the second aspect, the infusion pump device comprises a first air in line sensor, in particular an air in line ultrasound sensor, provided at said third tube portion.

According to a further preferred embodiment of the second aspect, the infusion pump device comprises a downstream pressure sensor provided at said fourth tube portion.

According to a further preferred embodiment of the second aspect, the infusion pump device comprises an air eliminating filter which is in fluid communication with the outlet of said peristaltic infusion pump tube segment and a second air in line sensor adapted to detect air in a fluid path downstream of said air eliminating filter.

According to a third aspect of the present invention, there is provided a medication reservoir for the infusion pump device of the second aspect, made of a single or multiple layered barrier polypropylene foil and sterile-packaged in a nitrogen filled aluminium bag.

So, according to a preferred embodiment of the present invention, the pump uses a linear peristaltic structure reducing friction with a ball bearing at each follower. The total construction of the infusion mechanism results in a small size weight and friction while reducing noise to minimum. The followers preferably comprise spring-loaded side walls, so that there is no kink at the sides during infusion, and are also subject to a removing action without friction by use of the same ball bearing resulting in squeezing the infusion tube through the same follower, wherein each follower comprises a runner at each side so as to slip without friction with side followers. In particular, the whole mechanism and motor assembly is included in one independent part comprising only one rod for assembly and a screw on the other side to adjust the distance from a pressure plate so as to operate with nominal pressure. In particular, the screw is adapted to adjust a squeezing gap in the infusion pump tube segment resulting in an adjustment of the maximum downstream occlusion pressure the infusion pump tube segment can withstand before leaking backwards, at nominal value. So, any contact points with the pump body are minimal, and also the motor and mechanism noise is not transmitted thought the body to the air, a very important feature for infusion pumps. According to a preferred embodiment, the mechanism has a silicone liquid ingress sealing membrane provided not over the followers as in prior art but at an intermediate point below their edge.

According to a preferred embodiment, the infusion segment is made by a relatively new silicon injection molding so that the accuracy tolerance is below 3% and is kept during all infusion time since silicone does not essentially change its form or shape after being squeezed by the followers. Injection molded silicon tubes have also a better accuracy than standard extruded silicon tubes showing large dimensional variations during extrusion.

According to a preferred embodiment, since an injection silicon is used that is formed by a mold, the infusion pump tube segment is provided with several tube portions which are better adapted to each function as follows:
Upstream there is a reduced thickness tube portion so that an upstream pressure sensor can sense even the smallest upstream occlusion (resulting in a reduction of this tube portion) since it is important to sense it as early as possible.
Subsequently there is a second normal infusion portion having thicker walls to increase the accuracy with higher suction so that it is reformed when the follower is removed, and to eliminate the need of a squeezing spring plate normally used in infusion pumps with a PVC infusion segment. This tube portion is not polished to reduce friction and power consumption.
Subsequently there is a third tube portion comprising a polished square section with flat outer walls for a better and secure air in line ultrasound reading, wherein ultrasound plates are best coupled to the tube.
Subsequently there is fourth tube portion including a flat bottom compartment having the shape of a larger diameter disc that when pressurized exercises a higher force to a downstream pressure sensor against it (F=P*S), a feature that is needed to sense accurately the downstream pressure from where important observations like the possibility of occlusion in a central venous catheter used by chronic patients are deduced.

Preferably, the wall of said first tube portion can have a thickness of about 0.5 mm, whereas the wall of said second tube portion can have a thickness of about 1.0 mm.

According to a further preferred embodiment, said first tube portion has an inner diameter of about 2.5 mm and an outer diameter of about 4 mm, said second tube portion has an inner diameter of about 2 mm and an outer diameter of about 4 mm and said third portion has an inner diameter of about 2 mm and an outer width of about 4 mm.

Preferably, the infusion pump tube segment comprises at least partly a shore hardness of about 50.

The pump preferably comprises means to reduce false alarms for air in line which are very often in hospital and home settings and are very annoying. A further preferred embodiment of the present invention uses a first air in line sensor downstream the pumping mechanism on the infusion pump tube segment so to sense the absence of liquid which leads to a bag empty alarm and a second air in line sensor downstream of an air eliminating filter put inside the drug compartment, so to eliminate the alarm generated by the first air in line sensor if no air is detected downstream and only bubbles but not full air is present at the first air in line detector. Also, preferably, it is detected if a filter is defective, and alarms for the defective filter are generated, if bubbles are detected by the second detector.

### BRIEF DESCRIPTION OF THE DRAWINGS

FIGS. 1a and 1b show a perspective top view (a) and a perspective side view (b) of a pump according to a preferred embodiment.

FIG. 2 shows the pump of FIG. 1 with its drug bag compartment being opened.

FIG. 3 shows a portion of the pump of FIG. 1 with its battery and GSM SIM card compartment.

FIGS. 4a and 4b show a peristaltic infusion pump tube segment to be provided within the pump of FIG. 1 in a perspective view (a) and a cross-sectional perspective view (b).

FIG. 5 shows a perspective view of a pump mechanism module as part of the pump of FIG. 1.

FIGS. 6a and 6b show as a part of the pump mechanism module of FIG. 5 a follower unit comprising a support with a cam and a follower movably provided at the support and including a ball bearing (a) and said follower individually shown from the opposite side (b).

FIG. 7 shows an embodiment of a drug bag with an RFID tag prefilled by a pharmaceutical company and including e.g. analgesics.

### DESCRIPTION OF A PREFERRED EMBODIMENT

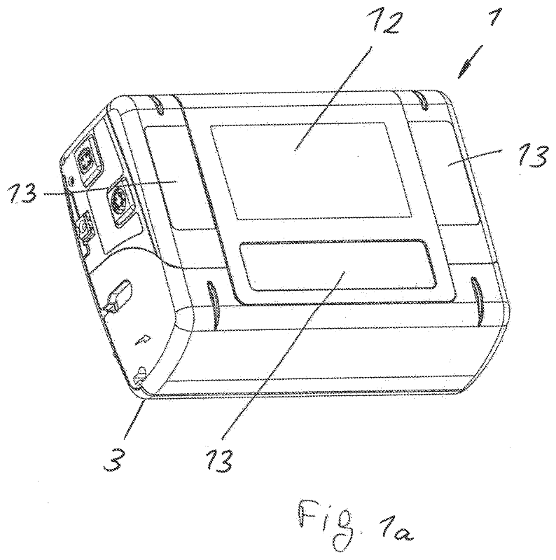

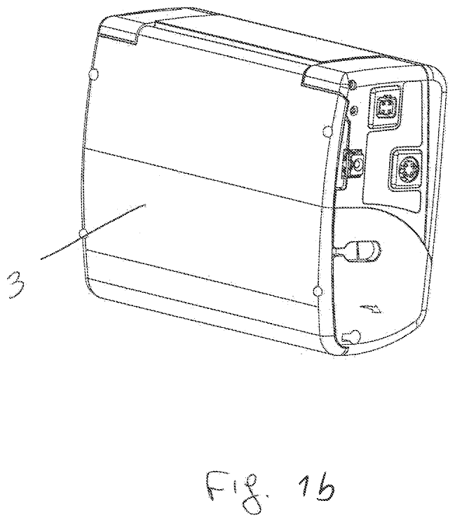

The pump 1 according to a preferred embodiment as shown in the FIGS. 1 to 3 includes a removable and exchangeable clear plastic drug bag compartment 4 with a lid 3 lockable by a key mechanism 7, and is embodied in several sizes depending on the therapy used so as to keep the size minimal for each therapy.

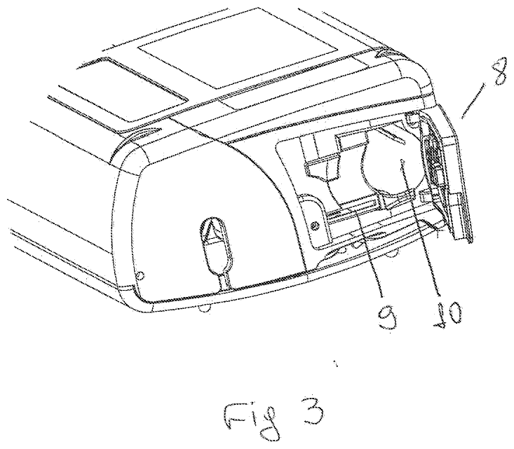

As to be seen from FIG. 3, the pump 1 comprises a battery compartment 10 with a disposable or rechargeable main battery and an internal rechargeable secondary battery (not shown). Two alkaline batteries in series are used as standard, but alternatively e.g. a rechargeable LiPo battery pack with separate connecting pins can be used (not shown), so that it is not possible to recharge alkaline batteries and, hence, to avoid errors. A battery door 8 when open allows access to a SIM card 9 for a GSM communication module (not shown).

The pump 1 additionally includes a smaller safety rechargeable battery (not shown), so that the pump 1 can even run during a battery change and alarm when the main battery has been depleted. This secondary battery is recharged by the main battery or an external power pack cable which is to be connected to the pump 1 and also charges the battery pack, while an external charger for replacement battery packs also exists.

The pump 1 includes a dual microcontroller electronic control for redundancy safety (not shown), and, as schematically depicted in FIG. 1a, further comprises an LCD display 12 and keys 13 with backlight, wherein the dual microcontroller electronic control is further adapted to control said display 12 and said keys 13 and optionally further components not shown.

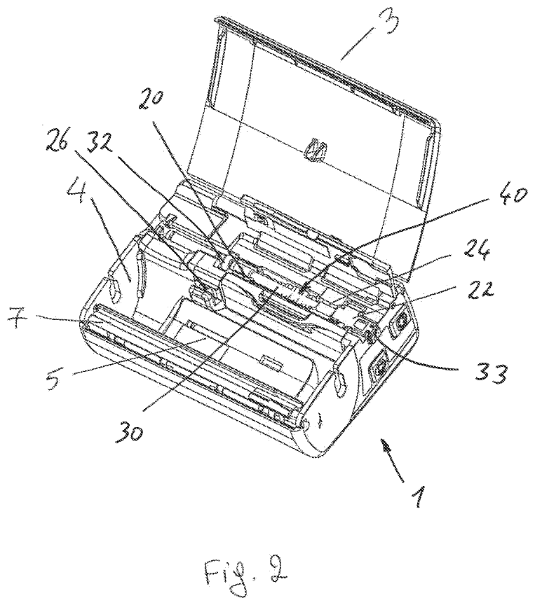

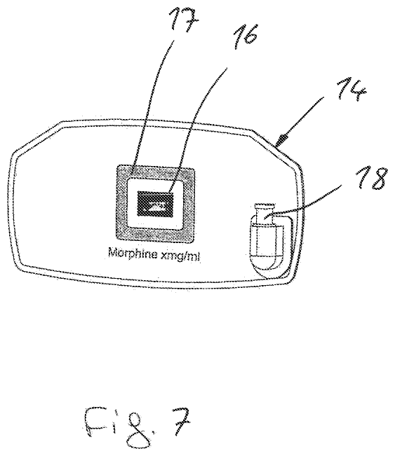

As also shown in FIG. 2, the pump 1 further comprises an RFID reader 5 at the bottom of the bag compartment 4, so that any tagged bag 14, as exemplarily shown in FIG. 7, which bag is provided with a tag or label 16 and an outlet 18 and is put into the compartment 4, can be read so as to make an infusion protocol selection automated and exclude medication errors. According to a system disclosed in EP 2 659 923 A1, an analgesia bag prefilled by a pharmaceutical company for this pump 1 is read automatically with the drug name and concentration guiding the pump 1 to find the correct protocol in an embedded or cloud-based drug library, by passing several steps of programming making it easy for nurses at home who are not trained on this pump 1 to perform an infusion excluding errors in preparation and programming, wherein there may be also the need of a second nurse (mandatory for safety in some countries).

According to the preferred embodiment shown in FIG. 7, the tag 16 on the pharmaceutical bag 14 is put (not with adhesive but) inside a pocket 17 formed by the same bag material, which pocket is thermally bonded on the bag material enclosing the tag with its antenna. The bag 14 is printed with written contents externally as usual on the flip side as also to be seen from FIG. 7. This solution is advantageous to avoid contamination of the drug with adhesive glue material. The outlet 18 of the drug bag 14 preferably includes a valve (not shown) which is opened by an infusion set connector (not shown), so that the drug bag 14 sold through pharmacies needs an infusion set sold through a pump 1 distribution network so as to connect with its outlet 18, also having infusion segment and an downstream accessories like filters and extension line tubing. This is a drug-device combination for a pharmaceutical product dedicated to a specific pump.

In order to help verify infusion and alarm conditions, the pump 1 includes an upstream pressure sensor 20 and a downstream pressure sensor 22, as to be seen from FIG. 2. As further shown in FIG. 2, an infusion pump segment 30 is inserted into the pump 1. The infusion pump segment 30 is disposable and forms a fluidic path from the outlet 18 of the drug bag 14 (FIG. 7) when inserted into the drug bag compartment 4 to an outlet. The infusion pump tube segment 30 comprises an upstream inlet 32 which is adapted to be coupled to the outlet 18 of the drug bag 14 by means of a fluid connector or connecting tubing (not shown) and further comprises a downstream outlet 33 for outputting a fluid drug or medication from the drug bag 14. As further to be seen from FIG. 2, the infusion pump tube segment 30 is to be arranged so that the upstream pressure sensor 20 is provided near the inlet 32 of the infusion pump tube segment 30 and the downstream pressure sensor 22 is provided near the outlet 33 of the infusion pump tube segment 30.

As further shown in FIG. 2, the pump 1 comprises a pump mechanism module 40 which is configured as a linear peristaltic mechanism and adapted to engage a portion of the infusion pump tube segment 30 so as to generate at least one squeezed point in said tube portion and to move it in the direction towards the outlet 33 of the infusion pump tube segment 30. So, the infusion pump tube segment 30 is to be arranged within the pump 1 so as to be subject by engagement of the pump mechanism module 40.

The pump 1 comprises means to reduce false alarms for air in line; these are very often in hospitals and home settings and are very annoying. In the embodiment shown in FIG. 2, said means comprise an air in line detector or sensor 24 downstream of the pump mechanism module 40 at the infusion pump tube segment 30 so as to sense the absence of liquid causing a bag empty alarm, and an additional air in line detector or sensor 26 after an air eliminating filter (not shown) arranged inside the drug compartment 4, so as to eliminate the alarm generated by said air in line sensor 24 if no air is detected downstream and only bubbles but not full air is present at said air in line sensor 24. The additional air in line sensor 26 is provided for detecting air after the air eliminating filter in a connecting tubing (not shown) to be positioned in the drug compartment 4 and connecting the outlet 18 of the drug bag 14 to the inlet 32 of the infusion pump tube segment 30 in some models for therapies like parenteral nutrition which usually generate a lot of air bubbles caused by an emulsion. There is also a detection if a filter is defective (not shown), and in said case alarms are given for a defective filter if bubbles are detected by the additional air in line sensor 26 preferably provided as mentioned above.

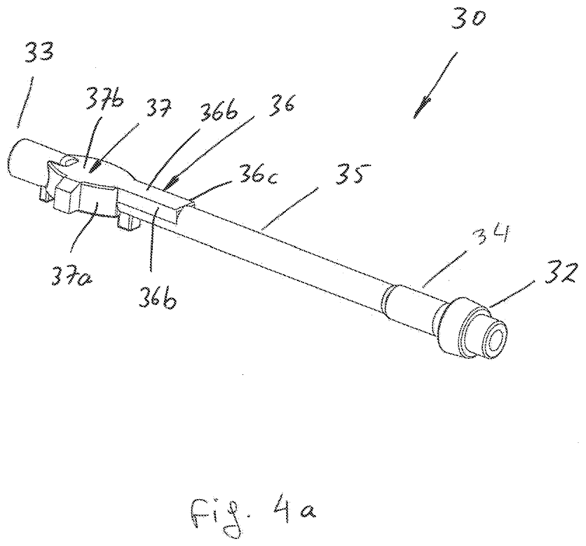

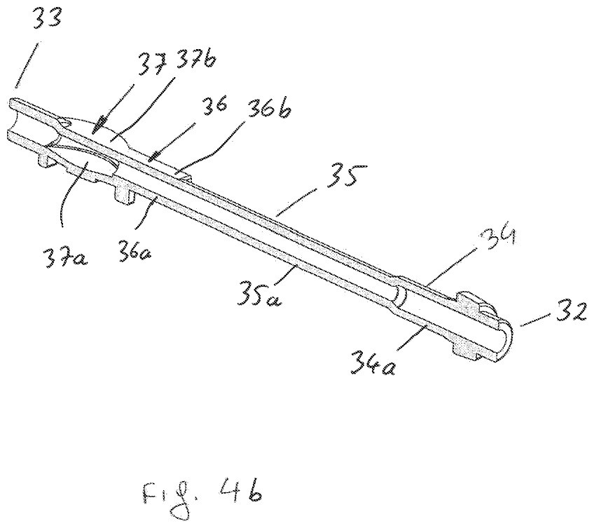

The infusion pump tube segment 30, which is individually shown in FIGS. 4a and 4b in greater detail, is preferably made by the relatively new silicon injection molding production so that the accuracy tolerance is below 3% and its shape and form are kept during all infusion time since silicone does not change when squeezed by the follower heads 42a. Injection molded silicon tubes have also a better accuracy than standard extruded silicon tubes, that have large dimensional variations during extrusion.

Since in particular the injection silicon formed by a mold is used, the infusion pump tube segment 30 comprises several portions 34 to 37 that are adapted to a certain function as follows:
Upstream, next to the inlet 32, there is a first tube portion 34 having a surrounding wall 34a with a reduced thickness (e.g. 0.5 mm) so that the upstream pressure sensor 20 can sense even the smallest suction in case of an upstream occlusion (reduction of the tube section) which is important to be sensed as early as possible.
Subsequently, there is a normal second tube portion 35 having a thicker wall 35a (e.g. 1.0 mm) with an increased accuracy by higher suction so that it is reformed when the follower 42a is removed, so as to eliminate the need of a squeezing spring plate usually used in infusion pumps with an infusion pump tube segment made of PVC. This second tube portion 35 is not polished to reduce friction and power consumption.
Subsequently, there is a third tube portion 36 having a polished square outer section 36c with flat walls 36b for a better and secure reading by air in line ultrasound sensors, as flat ultrasound plates engender best the flat sides of the tube.
Subsequently, next to the outlet 33, there is a fourth tube portion 37 including a flat bottom compartment 37a having the shape of a disc with a larger diameter which compartment when pressurized exercises a higher force to the downstream pressure sensor 22 against it (F=P*S), a feature needed to sense accurately the downstream pressure from where important observations like the possibility of occlusion of the central venous catheter used by chronic patients are deduced.

An infusion pump infusion set anti-kicking tubing is connected through barb connectors (not shown) to the infusion pump tube segment 30 at each side, i.e. at its inlet 32 and at its outlet 33. Furthermore, said compartment 37a includes a small seed (not shown) inserted inside its cavity to reduce high pressure variations and oscillations and to increase accuracy wherein that part has a hard cover on the top for better pressure reading but also to secure a barb connector aside it.

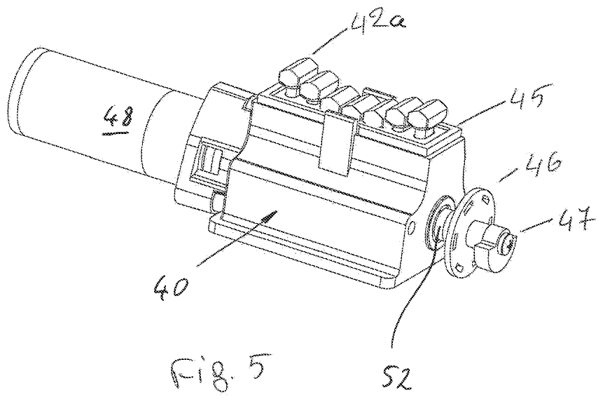

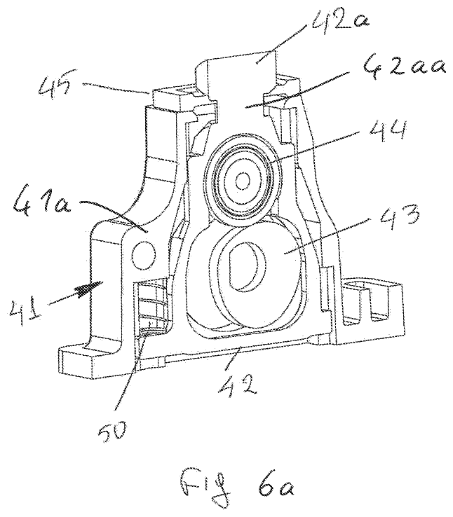

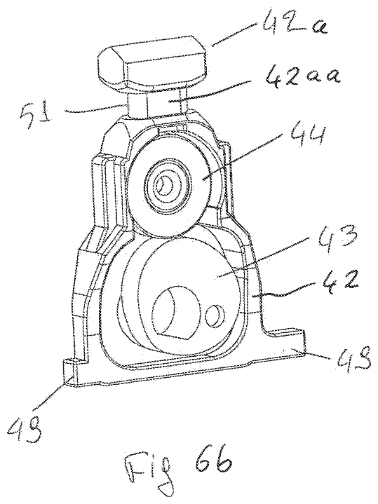

In the shown embodiment, the pump mechanism module 40, which is individually shown in FIG. 5 in greater detail, operates according to the linear peristaltic principle as basically known from EP 0 858 812 A1, and comprises a lot of follower units 41 arranged side by side. A preferred embodiment of a follower unit 41 is individually shown in FIG. 6a, and a follower which is part of the follower unit 41 is individually shown in FIG. 6b in greater detail wherein also the position of the rotating cam 43 inside it is also shown. The follower units 41 of the pump mechanism module 40 each operate with a reduced friction by means of a ball bearing 44 at a follower body 42 against an associated cam 43 which is arranged at a support 41a being part of the follower unit 41, wherein the follower body 42 is moveably mounted at said support 41a and comprises a follower head 42a provided on top of the follower body 42 (cf. FIGS. 6a and b). The cam 43 rolls over the ball bearing 44 for low friction and, this way, the cam 43 can have a form to reduce noise and also causes a uniform load on a motor 48 shown in FIG. 5 which motor is a drive unit for the pump mechanism module 40. The cam 43 of each follower unit 41 is mounted on a common rotary shaft 52 (a part of which is shown in FIG. 5) which is rotated by the motor 48, wherein the rotational angle offset of the cam 43 relative to the rotary shaft 52 increases from follower unit 41 to follower unit 41 so that the follower heads 42a are temporally brought into engagement with the infusion pump tube segment 30 from follower unit 41 to follower unit 41.

The total design of the pump mechanism module 40 is for small size weight and friction while reducing noise to minimum. The follower bodies 42 comprise spring loading side walls so that they do not kick at the sides during infusion (noise reduction) and perform a removing follower action without friction using the same ball bearing 44 that causes the squeezing of the infusion tube segment 30 through the same follower head 42a. Each follower body 42 is provided with a runner 49 at each side where it slips without friction with the neighboring follower body 42 as to be seen from FIG. 6b. There is a spring 50 between the support 41a and follower body 42 as shown in FIG. 6a in order to remove the follower head 42a from squeezing the infusion pump tube segment 30 in addition to a ball bearing action.

In case a follower body 42 is blocked, the cam 43 at the removing follower cycle (after having squeezed the infusion pump tube segment 30) will do it but with friction, as there is no ball bearing at the back side. This is why a spring force which is subject to the infusion pump tube segment 30 causes a removement of a follower body 42 without friction for small blocks, whereas the use of the cam 43 is the last means to force the infusion pump tube segment 30 to open but at a friction price to pay.

The pump mechanism module 40 as shown in FIG. 5 forms one independent part that has only one rod for assembly and a screw on the other side to adjust the distance from a pressure plate, resulting in an operation under nominal pressure. In particular, the screw is adapted to adjust a squeezing gap in the infusion pump tube segment resulting in an adjustment of the maximum downstream occlusion pressure the infusion pump tube segment can withstand before leaking backwards, at nominal value. This way as contact points with the pump 1 body are minimal, a motor and mechanism noise is not transmitted through the body to the air, being a very important feature for infusion pumps.

The mechanism has a silicone liquid ingress sealing membrane 45 provided (not over the follower heads 42a as in prior art but) at an intermediate point 51 below the edge of the follower heads 42a, as to be seen from the FIGS. 5 and 6a and b. The membrane 45 is preferably made by injection silicon molding and has orthogonal holes for a reduced section 42aa forming a stem and provided at each one of the follower bodies 42 at said point 51. The membrane 45 is very elastic and able to pass each follower head 42a from the hole water tightly, so that it is squeezed on the pump body by the pump mechanism module 40 for peripheral water tightness. A feedback for motor speed and infusion volume control is obtained by a first disc 46 for precise control (24 steps per turn) and a second disc 47 for rounds control also used as redundancy for safety control (cf. FIG. 5).

1. A peristaltic infusion pump tube segment having an upstream inlet and a downstream outlet and adapted to be temporally squeezed at at least one squeezed point to be generated by an engagement structure and to be moved towards said outlet for passing a medical fluid from said inlet to said outlet, characterized by:
a first tube portion next to said inlet and having a surrounding wall which is adapted for an upstream occlusion detection,
a second tube portion downstream of said first tube portion and adapted to be temporally engaged by the engagement structure, wherein said second tube portion comprises a surrounding wall being at least partly thicker than the wall of said first tube portion and preferably made of an elastic material which allows said second tube portion to be essentially reformed at those portions which are currently not subject to the engagement by the engagement structure,
a third tube portion downstream of said second tube portion and having a surrounding wall which is adapted for an air in line detection, and
a fourth tube portion next to said outlet and adapted for a downstream pressure detection.

2. The peristaltic infusion pump tube segment according to claim 1, which is, at least partly, made of silicone, in particular by a silicone molding process, preferably by a silicone injection molding process.

3. The peristaltic infusion pump tube segment according to claim 1, wherein the wall of said first tube portion has a thickness which is dimensioned so as to allow a detection unit to detect even the smallest possible upstream occlusion.

4. The peristaltic infusion pump tube segment according to claim 1, wherein the wall of said second tube portion is configured to allow a viscous fluid to be passed through by suction effect.

5. The peristaltic infusion pump tube segment according to claim 1, wherein the wall of said second tube portion is essentially unpolished.

6. The peristaltic infusion pump tube segment according to claim 1, wherein the wall of said third tube portion comprises at least one flat wall portion which is configured for the air in line detection in particular by means of at least one ultrasound sensor.

7. The peristaltic infusion pump tube segment according to claim 1, wherein said third tube portion has a rectangular or square section at least at its external side and comprises flat wall portions configured for the air in line detection in particular by means of at least one ultrasound sensor.

8. The peristaltic infusion pump tube segment according to claim 6, wherein the outer surface of the at least one flat wall portion is at least partly polished.

9. The peristaltic infusion pump tube segment according to claim 1, wherein said fourth tube portion comprises an essentially flat compartment configured for the downstream pressure detection.

10. The peristaltic infusion pump tube segment according to claim 9, wherein said flat compartment comprises a long side extending in the direction of the peristaltic infusion pump tube segment.

11. The peristaltic infusion pump tube segment according to claim 9, wherein said flat compartment comprises a disc-like element having essentially an at least part-circle or part-elliptical shape with a diameter or width being larger than the diameter or width of said first tube portion and/or said second tube portion (35) and/or said third tube portion (36).

12. An infusion pump device comprising a pump mechanism configured as a peristaltic mechanism and including the peristaltic infusion pump tube segment according to claim 1, wherein the upstream inlet of said peristaltic infusion pump tube segment is adapted to be fluidly connected to an outlet of a medication reservoir.

13. The infusion pump device according to claim 12, wherein said pump mechanism is configured as a linear peristaltic mechanism with said peristaltic infusion pump tube segment having an essentially elongated shape.

14. The infusion pump device according to claim 12, wherein said peristaltic infusion pump tube segment is replaceable and/or disposable.

15. The infusion pump device according to claim 12, comprising an upstream pressure sensor provided at said first tube portion.

16. The infusion pump device according to claim 12, wherein said pump mechanism comprises an engagement structure adapted to generate at least one squeezed point in said second tube portion and to move it in the direction towards said outlet of said peristaltic infusion pump tube segment.

17. The infusion pump device according to claim 13, wherein said engagement structure comprises a plurality of engagement units arranged side by side along the length of said second tube portion of said peristaltic infusion pump tube segment, wherein each engagement unit comprises a follower head which is movable at an angle, in particular a right angle, relative to said second tube portion and adapted to be brought into engagement with said second tube portion in order to squeeze it, wherein said engagement structure is further configured so that the follower heads are temporally brought into engagement with said second tube portion one after another.

18. The infusion pump device according to claim 17, wherein at least one engagement unit comprises a support, a follower body moveably mounted at said support and provided with the follower head, a ball bearing rotatably mounted at said follower body, a cam rotatably mounted at said support and in sliding engagement with said ball bearing, and a spring biasing said moveable follower body with said follower head away from said second tube portion of said peristaltic infusion pump tube segment.

19. The infusion pump device according to claim 18, wherein said engagement structure comprises a drive unit, wherein the cam of each equipment unit is fixedly mounted to a common rotary shaft which is rotated by said drive unit, and wherein the rotational angle offset of the cam relative to said rotary shaft increases from engagement unit to engagement unit so that the follower heads are temporally brought into engagement with said second tube portion of said peristaltic infusion pump tube segment from engagement unit to engagement unit.

20. The infusion pump device according to claim 18, wherein the follower head is mounted upon a stem fixed to the follower body and the engagement unit further comprises a sealing membrane with an opening through which said stem extends in an at least essentially sealing manner.

21. The infusion pump device according to claim 12, comprising a first air in line sensor, in particular an air in line ultrasound sensor, provided at said third tube portion.

22. The infusion pump device according to claim 12, comprising a downstream pressure sensor provided at said fourth tube portion.

23. The infusion pump device according to claim 12, comprising an air eliminating filter which is in fluid communication with the outlet of said peristaltic infusion pump tube segment and a second air in line sensor adapted to detect air in a fluid path downstream of said air eliminating filter.

24. A medication reservoir for the infusion pump device according to claim 16, made of a single or multiple layered barrier polypropylene foil and sterile-packaged in a nitrogen filled aluminium bag.
